(19) United States
(12) Patent Application Publication
(10) Pub. No.: US 2023/0346853 A1
(43) Pub. Date: Nov. 2, 2023

(54) Probiotic Lyophilizate Reactivation Composition for Improving Intestinal Survivability and Adhesion of Probiotics

(71) Applicants: COSMAX NBT, INC., Seongnam-si (KR); HEM PHARMA INC., Pohang-si (KR); COSMAX NS, INC., Seongnam-si (KR)

(72) Inventors: Jin Hak KIM, Seoul (KR); Su Jin KIM, Seoul (KR); Su Jeong KIM, Hanam-si (KR); Yosep JI, Suwon-si (KR)

(21) Appl. No.: 17/610,600

(22) PCT Filed: Mar. 29, 2021
(86) PCT No.: PCT/KR2021/003850

§ 371 Date: Nov. 11, 2021

(30) Foreign Application Priority Data
Jan. 20, 2021 (KR) 10-2021-0008188

(51) Int. Cl. A61K 35/742; C12N 1/20; A61K 9/19

(52) U.S. Cl. CPC A61K 35/742; C12N 1/20; A61K 9/19

(57) ABSTRACT

Disclosed herein is a Zeta-bio composition for reactivating lyophilized probiotics by conferring a negative zeta-potential on the cell surface of the probiotics, the composition comprising as a reactivator at least one selected from the group consisting of L-lysine, L-ornithine, L-tyrosine, and L-histidine. The composition activates lyophilized probiotics by conferring a negative zeta-potential thereon, with the consequent improvement of intestinal survivability and adhesion in the lyophilized probiotics in addition to exhibiting a restorative effect from cell damage due to lyophilization.

### TECHNICAL FIELD

The present disclosure relates to a composition that rehydrates and activates lyophilized probiotics within a short time, and a method for activation of lyophilized probiotics.

### BACKGROUND

Probiotics are known to have a beneficial impact on host health when administered in adequate amounts. Scientific evidence is steadily being accumulated on the beneficial impact of probiotics on human health in various ways including the alleviation of immune disorders, inflammatory bowel disease, type 2 diabetes, atherosclerosis, etc. Although recommendations tend to favor the consumption of a high dose of probiotics, neither the specific dosage nor the minimal viable numbers required for a putative probiotic strain are well-defined.

While strains with potential probiotic properties can be “naturally” obtained via fermented food such as yogurt, the distribution of freeze-dried probiotic powders packaged in sachets or capsules is also rapidly expanding in the market. Marketed probiotics should be transportable, shelf-stable concentrates that guarantee the effects of its intrinsic functional properties at room temperature. Commercialization of non-dairy probiotic products require exact optimization of the final processing steps such as the freezing and drying process.

The freeze-drying (or lyophilization) process is considered an appropriate approach to guarantee an extended shelf life for most probiotic products. However, the process is also known to create a stressful condition for live bacteria. The freeze-drying process is indeed a “challenge” to the viability of probiotic strains. Therefore, in order to maintain an effective dose, the number of bacteria in most probiotic products are three- to ten-folds higher than the numbers stated on the product label.

Probiotics are, for the most part, fatally damaged in a freeze-drying process, and when exposed in such a damaged state to the gastric acid and bile in the human body, the probiotics further decrease in viability due to the fatal stress. Unless they effectively adhere to the intestine, even the probiotics that have survived to the intestine have difficulty in sufficiently exhibiting functional effects. Therefore, the key requirements for guaranteeing the efficacy of probiotics include their survival under the gastrointestinal stress conditions and their adhesion to the intestinal wall. Colon cell-lines may be used as an in vitro model for determining the adherence potential of a putative probiotic strain (non-patent document 1). Adhesion properties of the bacterial cell envelope are determined by surface characteristics such as hydrophobicity, extracellular polymers, and the electric charge. Concurrently, the cell membrane plays a key role in the maintenance of cellular homeostasis and the support of intracellular functions. While interacting with their environment, bacteria are exposed to diverse physical forces that are transmitted by the specific surface structures to the cell. Diverse functional acidic and basic groups such as phospholipids, lipopolysaccharides (LPS) on the membrane surface of Gram-negative bacteria and surface proteins, and hydrocarbon-like proteins such as lipoteichoic and teichoic acids on the cellular surface of Gram-positive bacteria determine a strain's response to its environment (non-patent documents 2 and 3).

It is important to preserve the cell membrane to maintain the cell viability and adhesive functionality, which are necessary for an effective potential probiotic. The cells have to survive gastric stress and attach properly to the intestinal cells in order to promote immunomodulatory and metabolic functions, help strengthen the gut barrier, and competitively inhibit the adhesion of pathogens. Most of the adhesive factors, such as lipoteichoic acid, surface layer proteins, mucous binding proteins, and the like, are located around the cell surface of the bacteria. Although the process of lyophilization helps maintain the bacterial shelf-life by the reduction of water activity, it also disrupts the cell membrane, which may lead to the loss of its original functionality (non-patent document 4). Recovering the integrity of the cell membrane may help reactivate its functionality through the increase in the viability and cell adhesive properties.

The electrostatic charge of the cell surface is considered to be a reflection of its functional groups. When in contact with a liquid, the surface charge of a bacterial cell can be measured in millivolt units as zeta or electrokinetic potential. Both the cell surface composition and the properties of the surrounding medium (e.g., conductivity/ionic strength and pH) determine the cell's zeta potential.

Recently, the zeta potential around bacteria has emerged as an important indicator of bacterial viability and integrity efficacy especially in terms of physiology. However, no reports have been made on the use of the zetapotential in cell reactivation by rehydrating probiotic lyophilizates.

(Non-patent document 1) Arellano K., Vazquez J., Park H., Lim J., Ji Y., Kang H., Cho D., Jeong H. W. and Holzapfel W. H. (2019). Safety evaluation and whole-genome annotation of Lactobacillus plantarum strains from different sources with special focus on isolates from green tea. Probiotics and Antimicrobial Proteins

(Non-patent document 2) Dufrene, Y.F. and Persat, A. (2020). Mechanomicroiology: how bacteria sense the respond to forces. Nat. Rev. Microbiol. 18: 227-240

(Non-patent document 3) Boonaert, C. J. P. and Rouxhet, P. G. (2000). Surface of lactic acid bacteria: relationships between chemical composition and physicochemical properties. Appl. Environ. Microbiol. 66:2548-2554.

(Non-patent document 4) Govender, M., Choonara, Y. E., Kumar, P., du Toit, L. C., van Vuuren, S., & Pillay, V. (2014). A review of the advancements in probiotic delivery: Conventional vs. non-conventional formulations for intestinal flora supplementation. Aaps PharmSciTech, 15(1), 29-43.

### SUMMARY

According to various embodiments thereof, the present disclosure provides a composition for reactivation of probiotics and a method for reactivation of lyophilized probiotics, wherein the lyophilized probiotics are activated within a short time and provided with improved survivability and intestinal adhesion by conferring a suitable negative zeta potential thereon, which results in maximizing probiotic efficacy.

According to other various embodiments thereof, the present disclosure provides a composition for activating lyophilized probiotics, whereby probiotic cells can be restored from the damage attributed to the stress of lyophilization.

According to additional various embodiments thereof, the present disclosure provides a screening method, wherein a substance conferring a negative zeta potential on the cell surface of lyophilized probiotics is selected and used as a reactivator for improving survivability and intestinal adhesion of the lyophilized probiotics.

Leading to the present disclosure, the present inventors conducted intensive and thorough research into the reactivation of probiotic lyophilizates within a short time, and found that a substance conferring a negative zeta-potential on probiotics can be used as a reactivator that activates lyophilized probiotics, increases intestinal survivability and adhesion of lyophilized probiotics, and restores lyophilized probiotics from lyophilization-caused cell damage. It was surprisingly observed that carbohydrates, amino acids, and proteins which function to preserve cell membranes, especially amino acids selected from the group consisting of L-lysine, L-ornithine, L-tyrosine, and L-histidine, can serve as very effective reactivators for reactivating various lyophilized probiotics by conferring negative zeta-potentials on the cell surface of the lyophilized probiotics.

The composition and method according to an aspect of the present disclosure have the effect of reactivating lyophilized probiotics and improving their functionality by conferring a suitable negative zeta potential on the cell surface of the lyophilized probiotics.

Having such effects, the composition and method of the present disclosure can improve the survivability and intestinal adhesion of lyophilized probiotics and restore the cell membranes from damage.

In addition, due to such effects, the composition and method of the present disclosure can achieve cost reduction and excellent efficacy in the probiotic market where the probiotic efficacy has only been guaranteed by inputting a tremendous number of lyophilized probiotics.

### BRIEF DESCRIPTION OF DRAWINGS

FIG. 1 is a plot showing zeta potentials of lyophilized Lactobacillus plantarum HAC03 added with nine amino acids individually.

FIG. 2 shows results of assays for adherence to intestinal cells in terms of bacterial counts of Lactobacillus casei Lc-11 (A), Bifidobacterium longum B1-05 (B), Lactobacillus plantarum Lp-115 (C), and 7-strain mix (400B) (D), which were in the form of fresh cells, lyophilized probiotics, L-lysine-activated lyophilized probiotics, or proline-mixed lyophilized probiotics; and in terms of relative adhesion ratio (%) (counts of attached bacteria in test groups to counts of attached bacteria in the control) of Lactobacillus casei Lc-11 (E), Bifidobacterium longum B1-05 (F), Lactobacillus plantarum Lp-115 (G), and 7-strain mix (400B) (H), which were in the form of fresh cells, lyophilized probiotics, L-lysine-activated lyophilized probiotics, or proline-mixed lyophilized probiotics.

FIG. 3 shows results of assays for adherence to intestinal cells in terms of bacterial counts of Lactobacillus plantarum Lp-115 (A) and 7-strain mix (400B) (C), which were in the forms of fresh cells, lyophilized probiotics, or lyophilized probiotics activated with the Zeta-bio compositions of the present disclosure and in terms of relative adhesion ratio (%) (counts of attached bacteria in test groups to counts of attached bacteria in the control) of Lactobacillus plantarum Lp-115 (B) and 7-strain mix (400B) (D), which were in the form of fresh cells, lyophilized probiotics, or lyophilized probiotics activated with the Zeta-bio compositions of the present disclosure.

### DETAILED DESCRIPTION

Embodiments of the present disclosure are illustrated for describing the technical spirit of the present disclosure. The scope of the claims according to the present disclosure is not limited to the embodiments described below or to the detailed descriptions of these embodiments.

All technical or scientific terms used herein have meanings that are generally understood by a person having ordinary knowledge in the art to which the present disclosure pertains, unless otherwise specified. The terms used herein are selected for only more clear illustration of the present disclosure, and are not intended to limit the scope of claims in accordance with the present disclosure.

The expressions “include”, “provided with”, “have” and the like used herein should be understood as open-ended terms connoting the possibility of inclusion of other embodiments, unless otherwise mentioned in a phrase or sentence including the expressions.

Expressions such as “consisting of” used in the present disclosure should be understood as a closed-ended term that excludes the possibility of including other configurations in addition to the corresponding configuration.

A singular expression used herein can include meanings of plurality, unless otherwise mentioned, and the same is applied to a singular expression stated in the claims.

In one aspect of the present disclosure, the term “about” is used for the purpose of including an error in the manufacturing process in the specific numerical value or a slight numerical adjustment that falls within the scope of the technical idea of the present disclosure. For example, the term “about” means a range of ±10% of the value it refers to, ±5% on one side, and ±2% on the other. In the field of this disclosure, an approximation of this level is appropriate unless the values are specifically stated to require a narrower range.

Below, a detailed description will be given of a composition according to an aspect of the present disclosure.

An aspect of the present disclosure pertains to a composition that may reactivate lyophilized probiotics to confer a proper negative zeta-potential on the cell surface of the probiotics. As a reactivator of lyophilized probiotics, the composition of the present disclosure can improve intestinal survivability and adhesion by reactivating lyophilized probiotics and conferring a proper negative zeta-potential on them rather than simply rehydrating probiotic lyophilizates. A zeta-potential can be used as an indicator for bacterial viability, and changes in the zeta-potential reflect membrane damage and alterations in permeability. Depolarized zeta-potentials account for intact cell membranes. Hence, the present inventors introduced for the first time the zeta potential concept into the reactivation of lyophilized probiotics, finding that alteration to a negative zeta potential brings about cell reactivation, recovery from damages, and increase in viability.

In an aspect of the present disclosure, the composition for reactivation of probiotics according to the present disclosure may comprise as a reactivator at least one selected from the group consisting of L-lysine, L-ornithine, L-tyrosine, and L-histidine, preferably L-lysine and/or L-tyrosine, and more preferably L-lysine.

In the present disclosure, L-lysine may be L-lysine hydrochloride.

In an aspect of the present disclosure, the composition of the present disclosure may contain a reactivator at a concentration of about 0.01 M to about 0.15 M, about 0.01 M to about 0.1 M, about 0.02 M to about 0.07 M, about 0.02 M to about 0.05 M, or about 0.03 M to about 0.05 M when the reactivator is dissolved in a solvent. When used at a concentration below the lower limit, the reactivator may not sufficiently activate lyophilized probiotics. At a concentration exceeding the upper limit, there are problems as the probiotics may die, a high-concentration product may be difficult to achieve, and the products comprising the composition of the present disclosure may become poor in taste.

In an aspect of the present disclosure, the composition for reactivation of probiotics according to the present disclosure may further comprise at least one carbohydrate selected from the group consisting of fructose, sucrose, sorbitol, glucose, maltose, trehalose, and fructooligosaccharide, preferably, fructooligosaccharide.

In the present disclosure, the at least one carbohydrate selected from the group consisting of fructose, sucrose, sorbitol, glucose, maltose, trehalose, and fructooligosaccharide performs not only a cryoprotective function for the lyophilization of probiotics, but also prevents the lyophilized probiotics from undergoing cell membrane damage caused by osmosis upon rehydration and increases intestinal viability of the probiotics. Preferable in the present disclosure is a carbohydrate that has no influence on the reactivator's function of conferring a negative zeta potential on the cell surface of the lyophilized probiotics.

In the present disclosure, fructooligosaccharide, which is a functional oligosaccharide, is similar in sweetness and physical properties to cane sugar and thus can improve the taste of a product containing the composition of the present disclosure. Serving as a prebiotic, fructooligosaccharide can prolong the shelf life, is stable to heat and acidity, and is significantly resistant to acid, proteases, and bile acid during passage through the gastrointestinal tract. Due to the resistance to the gastric stress, fructooligosaccharide can improve the viability of the probiotics. Furthermore, fructooligosaccharide can provide beneficial health effects in preventing weight-gain and intestinal diseases through the production of short chain fatty acids. Fructooligosaccharide also modulates the microbiome to a healthy state by increasing the ratio of beneficial bacteria such as Bifidus, etc. and decreasing harmful bacteria in the human gut. Particularly, when used in combination with the reactivator, which is an amino acid, fructooligosaccharide can enhance the resistance of the probiotics in an unbalanced osmotic condition.

In an aspect of the present disclosure, the composition of the present disclosure may comprise a carbohydrate in an amount of about 0.1 g to about 8 g, about 0.2 g to about 6 g, about 0.3 g to about 5 g, about 0.5 g to about 4 g, or about 1 g to about 4 g. When the content of the carbohydrate is below the lower limit, it is difficult to expect a synergistic effect on restoration of the damaged cell membrane upon rehydration of probiotic lyophilizates and to sufficiently increase intestinal survivability and adherence to intestinal cells.

In an aspect of the present disclosure, the composition of the present disclosure may comprise lyophilized probiotics at a concentration of about 1×10⁸ to about 1×10¹² CFU/g.

In an aspect of the present disclosure, the lyophilized probiotics that can be reactivated with the composition may be Lactobacillus sp., Lactococcus sp., Enterococcus sp., Bifidobacterium sp., Pediococcus sp., Streptococcus sp., or a combination thereof. In detail, the lyophilized probiotics of the present disclosure may be Lactobacillus plantarum, Lactobacillus acidophilus, Lactobacillus casei, Streptococcus thermophilus, Bifidobacterium animalis, Bifidobacterium longum, Bifidobacterium breve, Bifidobacterium lactis, Lactobacillus reuteri, Lactobacillus gasseri, Enterococcus faecium, Clostridium butyricum, Lactobacillus rhamnosus, Streptococcus thermophilus, Lactobacillus delbrueckii ssp. Bulgaricus, Lactobacillus helveticus, Lactobacillus fermentum, Lactobacillus paracasei, Lactobacillus salivarius, Lactococcus lactis, Enterococcus faecalis, Bifidobacterium bifidum, or a combination thereof.

In an aspect of the present invention, the composition may be reactivated ex vivo by dissolution in a solvent before the intake or in vivo by drinking a solvent just before or after the intake. In the present disclosure, the probiotics may have a negative zeta potential on the cell surface thereof when the lyophilized probiotics are reactivated.

In an aspect of the present disclosure, the lyophilized probiotics may be in a form of freeze-dried powders.

In an aspect of the present disclosure, the composition may further comprise an ingredient, such as an amino acid, e.g., L-glutamic acid, L-serine, L-threonine, L-tryptophan, L-phenylalanine, etc., betaine, taurine, riboflavin, thiamine, or the like, which are all able to increase the survivability of lyophilized probiotics by restoring the cell membrane. The additional ingredient may be contained in such an amount in the composition as to have a final concentration of 0.01 M to 0.15 M when the composition is dissolved in a solvent.

An aspect of the present disclosure pertains to a method for reactivation of lyophilized probiotics, wherein the composition of the present disclosure is dissolved in a solvent before intake to confer a negative zeta potential on the cell surface of the probiotics, whereby the lyophilized probiotics are reactivated. In an embodiment, the lyophilized probiotics may be reactivated by ingesting the composition of the present disclosure before or after the intake of a solvent. So long as it allows the rehydration of both the composition of the present disclosure and probiotic lyophilizates, any method may be employed, without limitations imparted thereto.

An aspect of the present disclosure pertains to a method for reactivation of lyophilized probiotics, the method comprising contacting the lyophilized probiotics with a reactivator including at least one selected from the group consisting of L-lysine, L-ornithine, L-tyrosine, and L-histidine.

In an aspect of the present disclosure, the solvent may be any drinkable solvent, without particular limitations imparted thereto, and may be preferably water.

In the method for reactivation of lyophilized probiotics according to the present disclosure, the composition of the present disclosure is dissolved in a solvent to activate probiotics within about 30 min, about 10 min, about 5 min, about 1 min, about 30 sec, or several seconds. Whether the lyophilized probiotics are activated within a short time can be determined by adding the composition of the present disclosure with an edible dye that changes in color with pH.

In an aspect of the present disclosure, the composition of the present disclosure may be mixed with a solvent in an amount of 0.1 to 20 times the weight of the composition, particularly in an amount of 0.5 to 15 times or in an amount of 1 to 10 times the weight of the composition. By way of example, the solvent may be used in an amount of 1 to 200 ml, 5 to 150 ml, or 10 to 100 ml per 10 g of the composition.

An aspect of the present disclosure pertains to a probiotic product comprising the composition of the present disclosure. In an aspect of the present disclosure, the probiotic product comprising the composition of the present disclosure may be provided in separate package forms of the lyophilized probiotics and the other ingredients or in an integrated package form of the lyophilized probiotics and the other ingredients. The probiotic product comprising the composition of the present disclosure may be provided in the form of a sachet or a capsule. In an aspect of the present disclosure, the composition of the present disclosure may be dissolved in a solvent to reactivate the lyophilized probiotics before intake or may be ingested just before or after the intake of a solvent to reactivate the lyophilized probiotics according to proper instructions.

An aspect of the present disclosure pertains to a method for screening a substance capable of reactivating lyophilized probiotics, the method comprising selecting a substance that confers a negative zeta potential on a cell surface of the lyophilized probiotics. The substance that confers a negative zeta potential on the cell surface of lyophilized probiotics can reactivate the lyophilized probiotics, help increase survivability and adherence to intestinal cells, and repair damaged cell membranes.

### EXPERIMENTAL EXAMPLE 1 SCREENING FOR INGREDIENT CAPABLE OF REACTIVATING LYOPHILIZED PROBIOTICS BY CONFERRING NEGATIVE ZETA POTENTIAL ON CELL SURFACE OF LYOPHILIZED PROBIOTICS

In this experimental example, Lactobacillus plantarum HAC03 (accession number: KCTC13242BP, the Korean Research Institute of Bioscience and Biotechnology) (hereinafter referred to as “HAC03”), which was described in Korean Patent Application No. 10-2017-0051574, was used as a probiotic strain.

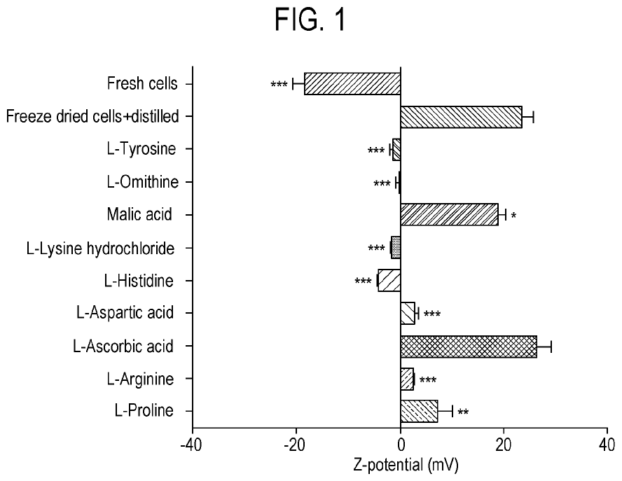

The lyophilized HAC03 strain powders were transferred at a concentration of 1×10⁹ CFU/g to a 50 mL tube and mixed with each of nine single amino acid ingredients (L-tyrosine, L-ornithine, malic acid, L-lysine, L-histidine, L-aspartic acid, L-ascorbic acid, L-arginine, and proline) to form a final concentration of 0.1 M. The mixtures of lyophilized HAC03 and the single ingredients were added with 1 mL of DW and rehydrated for 1 min. Afterwards, 2 mL of pH 2.5 distilled water was added to each sample, the pH was re-adjusted using HCl 0.1 N, and 800 μl of the calibrated sample was loaded in DTS1080 cuvettes. The electrophoretic mobility was measured by the Zetaizer Nano ZEN 3600 (Malvern Panalytica, UK) after 2 minutes of equilibration and the Smoluchowski equation was used to convert the data into zetapotential values. The converted zeta potential values are depicted in FIG. 1.

In this experiment, zeta potentials were measured at a pH of 2.5 in consideration of the highly acidic stomach environment, which is one of the initial hurdles that drastically reduce the survivability of the bacteria. As shown in FIG. 1, after lyophilization of HAC03, the zeta-potential of the bacterial cell depolarized significantly compared to the freshly cultured cells. When the bacteria was mixed with each of the nine amino acid ingredients, L-tyrosine, L-ornithine, L-lysine, and L-histidine produced negative zeta potentials as in the freshy cultured HAC03. However, the five remaining ingredients L-arginine, L-aspartic acid, malic acid, L-ascorbic acid, and proline could not change the depolarized zeta potentials into negative values and instead exhibited positive zeta potentials.

### EXPERIMENTAL EXAMPLE 2 INCREASE IN INTESTINAL VIABILITY OF LYOPHILIZED PROBIOTICS BY INGREDIENTS CONFERRING NEGATIVE ZETA POTENTIALS ON CELL SURFACE

HAC03 strain and the eight ingredients used in Experimental Example 1 were evaluated for probiotic viability (acid resistance and bile resistance) using the in vitro Simulated Stomach Duodenum Passage (SSDP) according to Ji et al (Food control 31(2):467-473, 2013) with some modifications. The HAC03 strain was mixed with each of the single compositions and 1 mL of distilled water for 1 min. The compositions were formulated to have a final concentration of 0.1 M when dissolved in 1 ml of distilled water. Thereafter, 9 mL of distilled water adjusted to have a pH of 2.5 was added to each mixture. The pH was readjusted back to 2.5 in case of an increased pH due to the nature of some of the single ingredients (in order to not affect the survival of the probiotics). After adjusting the pH to 2.5, the tubes were incubated at 37° C. for 1 hour to apply the low-pH gastric stress condition. Then, the strain was incubated for 2 hours under the condition of 4 ml of bile salts (10% ox gall)) and 17 ml of pH 6.0 synthetic duodenum juice (6.4 g/L NaHCO3, 0.239 g/L KCl, and 1.28 g/L NaCl), simulating the small intestine passage. During the gastrointestinal tract assay, the samples were taken immediately, 1 hour, and 3 hours after the incubation (t=0, 1, and 2). The probiotic survivability of each sample after gastric stress and bile stress was calculated by counting viable colonies. The measurements are summarized in Table 1, below.

TABLE 1

| | Initial | Stomach | | Duodenum | |
|---|---|---|---|---|---|
| | log | log | Survivability | | Survivability |
| Sample | CFU/mL | CFU/mL | (%) | log CFU/mL | (%) |
| Fresh bacteria | 8.46 ± 0.32 | 8.07 ± 0.65 | 54.26 | 7.43 ± 1.10 | 20.58 |
| Probiotic | 8.34 ± 0.34 | 7.16 ± 0.60 | 8.03 | 6.79 ± 0.60 | 3.40 |
| lyophilzate + | | | | | |
| distilled water | | | | | |
| L-Arginine | 6.88 ± 0.04 | 4.00 ± 0.00 | 0.13 | 3.73 ± 0.34 | 0.08 |
| L-Ascorbic acid | 6.49 ± 0.05 | 4.08 ± 0.15 | 0.40 | 3.49 ± 0.00 | 0.10 |
| L-Aspartic acid | 7.84 ± 0.07 | 7.06 ± 0.56 | 29.95 | 5.80 ± 0.64 | 1.86 |
| L-Histidine | 8.09 ± 0.05 | 7.83 ± 0.11 | 56.61 | 7.52 ± 0.08 | 27.20 |
| L-Lysine | 7.94 ± 0.04 | 7.87 ± 0.07 | 85.17 | 7.62 ± 0.09 | 48.35 |
| hydrochloride | | | | | |
| Malic acid | <4.99 ± 0.00 | <3.99 ± 0.00 | <0.01 | <3.49 ± 0.00 | <0.003 |
| L-Ornithine | 7.97 ± 0.04 | 7.75 ± 0.02 | 60.53 | 7.57 ± 0.03 | 40.14 |
| L-Tyrosine | 8.12 ± 0.03 | 7.98 ± 0.04 | 73.52 | 7.65 ± 0.01 | 33.89 |

As is understood from the data of Table 1, when incubated with the four ingredients (L-ornithine, L-lysine, L-tyrosine, and L-histidine) that conferred negative zeta potentials, the lyophilized probiotics increased in acid resistance and bile resistance and thus exhibited improved intestinal survivability, compared to fresh bacteria. In contrast, the four ingredients (arginine, ascorbic acid, aspartic acid, and malic acid) which could not shift the zeta potentials to negative values gave probiotic lyophilizates intestinal survivability far inferior to that of distilled water (survivability 3.40%). These data indicate that a substance conferring a negative zeta potential on the cell surface of lyophilized probiotics has a significant positive effect on the intestinal survivability of lyophilized probiotics.

### EXPERIMENTAL EXAMPLE 3 ASSAY FOR INTESTINAL ADHESION OF INGREDIENT CAPABLE OF REACTIVATING LYOPHILIZED PROBIOTICS

Among the four ingredients that conferred negative zeta potentials and increased intestinal survivability in lyophilized probiotics, L-lysine, which exhibited the highest survivability in Experimental Example 2, was used to reactivate lyophilized probiotics to assess the effect on cell adhesion to the intestine. An examination was made to see whether the effect of reactivating lyophilized probiotics is applicable to various strains in this experimental example. In this regard, lyophilized Bifidobacterium longum Bl-05 (Dupont; hereinafter referred to as “Bl-05”), Lactobacillus plantarum Lp-115 (Dupont; hereinafter referred to as “Lp-115”), Lactobacillus casei Lc-11 (Dupont; hereinafter referred to as “Lc-11”), and 7-strain mixture (400B) (Dupont; hereinafter referred to as “MIX”) were reactivated with L-lysine and then assayed for their ability to adhere to the human enterocyte-like Caco-2/TC-7 cell line. The 7-strain mix refers to a mixture of the 7 strains listed in Table 2, below. In this experimental example, proline, which maintains positive zeta potentials on the cell surface of lyophilized HAC03, was used as a control.

TABLE 2

| Strain name | Strain included |
|---|---|
| 7-Strain mixture (400B) | L. plantarum Lp-115 |
| | L. acidophilus La-14 |
| | L. casei Lc-11 |
| | Streptococcus thermophilus St-21 |
| | Bifidobacterium animalis subsp. lactis Bl-04 |
| | B. longum Bl-05 |
| | B. breve Bb-03 |

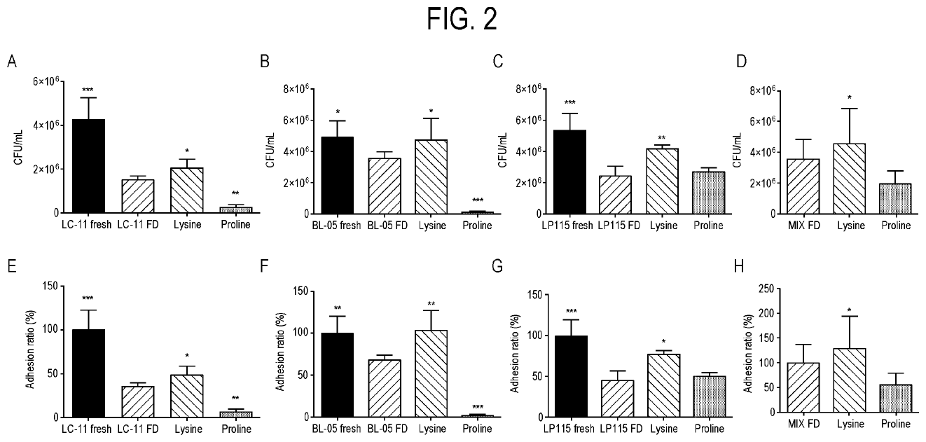

Lyophilizates of the three strains and the 7-strain mix were adjusted to 2×10⁸ CFU/g and mixed with 0.03 M L-lysine or 0.03 M proline. The pellets were washed three time with 1× PBS and resuspended in 10 ml MEM cell culture media supplemented with 20% FBS, 2 mM glutamine, and 1% non-essential amino acids. A bacterial adhesion assay was performed according to the method of Botes et al. (Arch. Microbiol, 190 (2008), pp. 573-584) with some modifications. To assess the bacterial cell adhesion, 1×10⁵ CFU/Caco-2/TC-7 monolayer was incubated with 2 mL of each bacterial suspension at 37° C. for 1.5 hours in an atmosphere of 5% CO₂ and 95% air. The cells were washed three times with cold PBS to remove non-attached bacteria and lysed for 15 min at 37° C. by adding 40 ng of trypsin (Promega). To quantify the number of bacteria attached to Caco-2/TC-7 cells, the samples were serially diluted, incubated on MRS agar plates for 48 hours (37° C.), and then counted to measure relative adhesion ratio (counts of attached bacteria in test groups to counts of attached bacteria in the control). The results are depicted in FIG. 2. All the experiments were conducted in triplicate.

As can be seen in FIG. 2, freeze-dried forms (FD) of Lc-11 (FIGS. 2A and 2E), B1-05 (FIGS. 2B and 2F), and Lp-115 (FIGS. 2C and 2G) had significantly lower attachment of bacterial cells to the Caco-2/TC-7 cell line compared to the fresh cells (fresh). Reactivation with L-lysine significantly increased bacterial cell adhesion compared to the freeze-dried form. This result was replicated in the 7-stain mixture (400B), as well (FIGS. 2D and 2H). However, the freeze-dried forms in mixture with proline (control), which confers positive zeta potentials on the cell surface of lyophilized probiotics, were remarkably lower in adhesion than the freeze-dried forms themselves (Lc-11, B1-05, MIX) and those reactivated with L-lysine (Lp-115). In other words, lyophilized probiotics increased in adhesion to the intestine when reactivated with a substance capable of conferring negative zeta potentials on the cell surface thereof such as L-lysine, whereas substances conferring positive zeta potentials exhibited no improved adhesion effects. Taken together, the data demonstrate that a negative zeta potential on a cell surface correlates with an improvement in adhesion to the intestine.

### EXPERIMENTAL EXAMPLE 4 ADDITIONAL INGREDIENTS USABLE TOGETHER WITH REACTIVATORS OF LYOPHILIZED PROBIOTICS

A test was conducted to examine ingredients that could be used together with reactivator ingredients capable of conferring negative zeta potentials on the cell surface of lyophilized probiotics. HAC03 lyophilizates were mixed with each of the 11 carbohydrates listed in Table 3, below and assayed for intestinal survivability in the same manner as in Example 1. The results are summarized in Table 3, below.

TABLE 3

| | | Artificial Gastric Juice | | Artificial Bile Juice | |
|---|---|---|---|---|---|
| | Initial | | Surviv- | | Surviv- |
| | log | log | ability | log | ability |
| Sample | CFU/mL | CFU/mL | (%) | CFU/mL | (%) |
| Fresh cell | 8.46 ± 0.32 | 8.07 ± 0.65 | 54.26 | 7.43 ± 1.10 | 20.58 |
| Lyophilized | 8.34 ± 0.34 | 7.16 ± 0.60 | 8.03 | 6.79 ± 0.60 | 3.40 |
| Arabinose | 8.10 ± 0.11 | 7.12 ± 0.67 | 17.78 | 7.02 ± 0.35 | 9.45 |
| Xylose | 8.08 ± 0.15 | 7.50 ± 0.25 | 27.36 | 6.97 ± 0.49 | 8.47 |
| Rhamnose | 8.27 ± 0.09 | 7.87 ± 0.10 | 40.75 | 7.53 ± 0.08 | 18.25 |
| Fructose | 8.05 ± 0.17 | 7.67 ± 0.37 | 46.18 | 7.27 ± 0.48 | 20.73 |
| Mannitol | 7.94 ± 0.05 | 7.18 ± 0.32 | 20.04 | 6.90 ± 0.44 | 11.77 |
| Sucrose | 8.08 ± 0.13 | 7.99 ± 0.13 | 80.90 | 7.71 ± 0.13 | 42.99 |
| Sorbitol | 8.08 ± 0.08 | 8.00 ± 0.05 | 82.06 | 7.69 ± 0.09 | 40.46 |
| Glucose | 8.05 ± 0.09 | 7.93 ± 0.11 | 76.99 | 7.64 ± 0.03 | 39.54 |
| Maltose | 8.09 ± 0.05 | 8.02 ± 0.05 | 85.43 | 7.61 ± 0.13 | 33.79 |
| Trehalose | 7.91 ± 0.10 | 7.65 ± 0.05 | 54.75 | 7.36 ± 0.03 | 28.10 |
| Fructooligosaccharide | 8.20 ± 0.12 | 7.99 ± 0.05 | 62.47 | 7.57 ± 0.11 | 23.52 |

As can be seen in Table 3, the seven ingredients fructose, sucrose, sorbitol, glucose, maltose, trehalose, and fructooligosaccharide allowed survivability similar to or higher than that of fresh cells and were observed to guarantee particularly higher bile resistance than that of fresh cells. Therefore, those ingredients can be used, together with the reactivator, to reactivate probiotic lyophilizates and increase their intestinal survivability.

### EXPERIMENTAL EXAMPLE 5 ASSAY FOR SURVIVABILITY AFTER REACTIVATION OF LYOPHILIZED PROBIOTICS WITH INVENTIVE COMPOSITION

An experiment was carried out in order to examine the effect of the composition of the present disclosure on reactivation of lyophilized probiotics. First, compositions of the present disclosure (hereinafter abbreviated to “Zeta-bio compositions”) were prepared to include L-lysine, fructooligosaccharide (FOS), and microorganisms (probiotics) according to the compositions listed in Table 4, below. Fructooligosaccharide was measured to have a zeta potential near 0 mV and thus does not affect the zeta potential of the lyophilized probiotics of the present disclosure. Lyophilized Lp-115 and MIX were used as lyophilized probiotics.

TABLE 4

| Ingredients | LP/Mix-1 | | LP/Mix-2 | | LP/Mix-3 | | LP/Mix-4 | | LP/Mix-5 | |
|---|---|---|---|---|---|---|---|---|---|---|
| L-Lysine | 0.183 g | | 0.365 g | | 0.548 g | | 0.731 g | | 0.913 g | |
| hydrochloride | (0.01M) | | (0.02M) | | (0.03M) | | (0.04M) | | (0.05M) | |
| fructooligosaccharide | 3.5 | g | 3.5 | g | 3.5 | g | 3.5 | g | 3.5 | g |
| Strain | 0.15 g | | 0.15 g | | 0.15 g | | 0.15 g | | 0.15 g | |
| | (2x1011 | | (2x1011 | | (2x1011 | | (2x1011 | | (2x1011 | |
| | CFU/g) | | CFU/g) | | CFU/g) | | CFU/g) | | CFU/g) | |
| Dextrin | 6.167 | g | 5.985 | g | 5.802 | g | 5.619 | g | 5.437 | g |
| Total | 10 | g | 10 | g | 10 | g | 10 | g | 10 | g |

The Zeta-bio compositions in Table 4 were assayed for probiotic survivability (acid resistance and bile resistance) by in vitro Simulated Stomach Duodenum Passage (SSDP) as in Experimental Example 2. The Zeta-bio composition in Table 3 were each mixed with 1 mL of distilled water at 25° C. for 1 min. In this regard, the Zeta-bio compositions were prepared to include L-lysine at a concentration of 0.01 M, 0.02 M, 0.03 M, 0.04 M, or 0.05 M when dissolved in 1 ml of distilled water. Then, 9 mL of distilled water adjusted to pH 2.5 was added to each mixture, and when increased due to the nature of some ingredients, the pH was readjusted back to pH 2.5 (in order to exclude any influence on survival of probiotics). The tubes were incubated at 37° C. for 1 hour to apply a low-pH gastric stress thereto. Subsequently, the tubes were exposed to 4 ml of bile salts (10% oxgall) and 17 ml of synthetic bile juice with pH 6.0 (6.4 g/L NaHCO3, 0.239 g/L KCl, and 1.28 g/L NaCl) for 2 hours to simulate the small intestine passage. During the gastrointestinal assay, samples were taken immediately 1 hour and 3 hours after the exposure (t=0, 1, and 2). The probiotic survivability of each sample after exposure to gastric stress and bile stress was calculated by counting viable colonies. The measurements are summarized in Table 5, below.

TABLE 5

| | | | Initial | Artificial Gastric Juice | | Artificial Bile Juice | |
|---|---|---|---|---|---|---|---|
| | | Lysine | log | log | Surviv- | 1og | Surviv- |
| Sample | Strain | Conc. | CFU/mL | CFU/mL | ability(%) | CFU/mL | ability(%) |
| Fresh cell | Lactobacillus | None | 8.21 ± 0.10 | 8.05 ± 0.26 | 64.45 | 7.61 ± 0.15 | 25.76 |
| Lyophilizate | plantarum | None | 8.04 ± 0.16 | 4.15 ± 0.16 | 0.01 | 4.02 ± 0.66 | 0.02 |
| LP-1 | Lp-115 | 0.01M | 7.05 ± 0.21 | 5.63 ± 0.05 | 5.07 | 4.29 ± 0.28 | 0.17 |
| LP-2 | | 0.02M | 8.46 ± 0.04 | 6.97 ± 0.01 | 4.25 | 6.26 ± 0.01 | 0.70 |
| LP-3 | | 0.03M | 8.59 ± 0.03 | 8.28 ± 0.08 | 48.64 | 7.73 ± 0.01 | 13.86 |
| LP-4 | | 0.04M | 8.62 ± 0.02 | 8.30 ± 0.00 | 48.01 | 7.69 ± 0.00 | 11.93 |
| LP-5 | | 0.05M | 8.63 ± 0.09 | 8.26 ± 0.01 | 43.20 | 7.72 ± 0.03 | 12.17 |
| Lyophilizate | 7 strain | None | 8.75 ± 0.02 | 7.52 ± 0.05 | 6.01 | 4.96 ± 0.07 | 0.02 |
| Mix-1 | mixture 8 | 0.01M | 8.20 ± 0.08 | 6.43 ± 0.00 | 1.74 | 5.88 ± 0.06 | 0.48 |
| Mix-2 | (400B) | 0.02M | 8.42 ± 0.01 | 7.79 ± 0.01 | 23.02 | 7.69 ± 0.04 | 18.70 |
| Mix-3 | | 0.03M | 8.32 ± 0.01 | 8.02 ± 0.01 | 50.09 | 7.94 ± 0.05 | 41.99 |
| Mix-4 | | 0.04M | 8.33 ± 0.03 | 7.95 ± 0.03 | 41.93 | 7.90 ± 0.01 | 37.65 |
| Mix-5 | | 0.05M | 8.37 ± 0.01 | 8.10 ± 0.01 | 53.33 | 7.82 ± 0.14 | 28.72 |

As can be seen in Table 5, lyophilized forms of Lp-115 remarkably decreased in survivability (0.02%), compared to the fresh cell control (fresh), after SSDP. When reactivated with the Zeta-bio compositions having 0.03 M or higher of L-lysine, the lyophilized Lp-115 exhibited survivability similar to or higher than that of fresh cells under the gastric stress and bile stress. Activation with the Zeta-bio composition having L-lysine at a concentration of 0.03 M or higher also guaranteed high survivability to the lyophilized 7-strain mixture (400B) under the gastric stress and bile stress. Therefore, probiotic lyophilizates activated with the Zeta-bio composition having 0.03 M or higher of L-lysine exhibit higher survivability after passage through the gastrointestinal tract compared to the lyophilized probiotics themselves.

### EXPERIMENTAL EXAMPLE 6 ASSAY FOR INTESTINAL ADHESION AFTER REACTIVATION OF LYOPHILIZED PROBIOTICS WITH INVENTIVE COMPOSITION

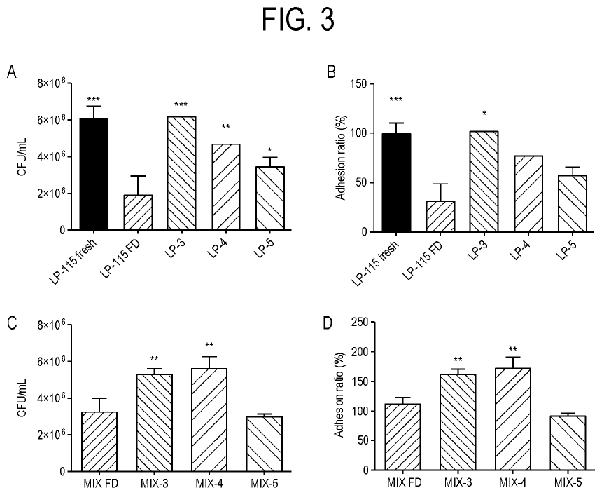

An experiment was carried out in order to examine the influence of the Zeta-bio compositions of the present disclosure on intestinal adhesion of lyophilized probiotics. The Zeta-bio compositions in Table 3 were observed to increase intestinal adhesion in lyophilized LP-115 and MIX when measured by the same assay for ability to adhere to the human enterocyte-like Caco-2/TC-7 cell line as in Experimental Example 3. The results are depicted in FIG. 3.

As can be seen in FIG. 3, the freeze-dried form (FD) of Lp-115 (FIGS. 3A and 3B) was significantly lower in bacterial cell adhesion to the Caco-2/TC-7 cell line than fresh cells (fresh). However, reactivation with the Zeta-bio compositions (LP-3 to LP-5) significantly recovered the bacterial cell adhesion, compared to the freeze-dried form. Lp-115 exhibited higher intestinal adhesion at all of the L-lysine concentrations tested, compared to the freeze-dried form. Also, the 7-strain mixture (400B) exhibited significantly high adhesion when reactivated with MIX-3 and MIX-4 compositions that contain L-lysine at concentrations of 0.03 M and 0.04 M, respectively. Therefore, the composition containing L-lysine according to the present disclosure can reactivate lyophilized probiotics and provide the lyophilized probiotics with increased survivability and intestinal adhesion.

### EXPERIMENTAL EXAMPLE 7 ASSAY OF LYOPHILIZED PROBIOTICS REACTIVATED WITH INVENTIVE COMPOSITIONS FOR SURVIVABILITY ACCORDING TO L-LYSINE CONCENTRATION

An experiment was carried out to examine the influences of the Zeta-bio compositions having L-lysine at a concentration of 0.1 M or higher as well as at a concentration of 0.01 M to 0.05 M as in Table 2 on survivability of lyophilized probiotics reactivated therewith. The Zeta-bio compositions used in this experimental example were identical to those of Table 2 in terms of configuration and content, but different in L-lysine concentration and were prepared to include L-lysine at a concentration of 0.1 M, 0.15 M, 0.3 M, 1.5 M, or 3 M when dissolved in 1 ml of distilled water for MIX-6, MIX-7, MIX-8. MIX-9, or MIX-10, respectively.

The Zeta-bio compositions were assayed for probiotic survivability (acid resistance and bile resistance) by in vitro Simulated Stomach Duodenum Passage (SSDP) as in Experimental Example 5. The measurements are summarized in Table 6, below.

TABLE 6

| | | | Initial | Artificial Gastric Juice | | Artificial Bile Juice | |
|---|---|---|---|---|---|---|---|
| | Lysine | | log | log | Survivability | log | Survivability |
| Strain | Conc. | Sample | CFU/mL | CFU/mL | (%) | CFU/mL | (%) |
| 7 Strain | None | Lyophilizate | 8.75 ± 0.02 | 7.52 ± 0.05 | 6.01 | 4.96 ± 0.07 | 0.02 |
| mixture | 0.03M | MIX-3 | 8.32 ± 0.01 | 8.02 ± 0.01 | 50.09 | 7.96 ± 0.01 | 41.31 |
| (400B) | 0.1M | MIX-6 | 8.44 ± 0.04 | 8.20 ± 0.01 | 57.08 | 7.51 ± 0.00 | 11.54 |
| | 0.15M | MIX-7 | 8.47 ± 0.01 | 8.34 ± 0.05 | 75.53 | 7.75 ± 0.02 | 19.12 |
| | 0.3M | MIX-8 | 8.69 ± 0.05 | 8.36 ± 0.05 | 47.06 | 3.49 ± 0.00 | 0.0006 |
| | 1.5M | MIX-9 | 9.01 ± 0.02 | 7.69 ± 0.04 | 4.83 | 3.49 ± 0.00 | 0.0003 |
| | 3M | MIX-10 | 9.03 ± 0.06 | 7.92 ± 0.03 | 7.81 | 3.49 ± 0.00 | 0.0003 |

As can be seen in Table 6, the compositions MIX-3, MIX-6, and MIX-7 containing L-lysine at respective concentrations of 0.03 M, 0.1 M, and 0.15 M exhibited significant survival rates of 57% or higher against gastric acid and 11% or higher against bile acid. It can therefore be understood from the data of Experimental Example 5 and this Experimental Example that lyophilized probiotics treated with a Zeta-bio composition having 0.01 to 0.15 M of a reactivator, such as L-lysine, which confers a negative zeta potential on the cell surface thereof, exhibit increased survivability after passage through the gastrointestinal tract, compared to the lyophilized probiotics themselves.

Rehydration of probiotic lyophilizates with the composition of the present disclosure confers a negative zeta potential to reactivate the cells, with the consequent increase of survivability therein. The composition of the present disclosure is considered to play a role in promoting repair of damaged cells by providing nutrients and essential cell components to injured cells. Moreover, the increase in viability and the alteration of zeta potential in cells may improve the adhesion ratio of the probiotics to intestinal cells. Consequently, the reactivation of lyophilized probiotic products with the Zeta-bio composition of the present disclosure can improve the viability of the probiotics even after exposure to the gastric and bile stresses and can restore their beneficial effects due to the increase of bacterial adherence to the intestinal cells.

Although the technical spirit of the present disclosure has been described by the examples described in some embodiments and illustrated in the accompanying drawings, it should be noted that various substitutions, modifications, and changes can be made without departing from the scope of the present disclosure which can be understood by those skilled in the art to which the present disclosure pertains. In addition, it should be noted that that such substitutions, modifications and changes are intended to fall within the scope of the appended claims.

1. A composition for reactivation of lyophilized probiotics, which confers a negative zeta-potential on cell surfaces of the probiotics to reactivate the lyophilized probiotics.

2. The composition of claim 1, wherein the composition comprises as a reactivator at least one selected from the group consisting of L-lysine, L-ornithine, L-tyrosine, and L-histidine.

3. The composition of claim 2, wherein the composition comprises L-lysine as the reactivator.

4. The composition of claim 2, wherein the reactivator is contained in such an amount as to form a final concentration of 0.01 M to 0.15 M when dissolved in a solvent.

5. The composition of claim 1, further comprising at least one carbohydrate selected from the group consisting of fructose, sucrose, sorbitol, glucose, maltose, trehalose, and fructooligosaccharide.

6. The composition of claim 5, wherein the carbohydrate is contained in an amount of 0.1 g to 8 g per 10 g of the composition.

7. The composition of claim 1, comprising lyophilized probiotics at a concentration of 1×10⁸ to 1×10¹² CFU/g.

8. The composition of claim 1, wherein the lyophilized probiotics are in a freeze-dried form of Lactobacillus sp., Lactococcus sp., Enterococcus sp., Bifidobacterium sp., Pediococcus sp., Streptococcus sp., or a combination thereof.

9. The composition of claim 8, wherein the lyophilized probiotics are in a freeze-dried form of Lactobacillus plantarum, Lactobacillus acidophilus, Lactobacillus casei, Streptococcus thermophilus, Bifidobacterium animalis, Bifidobacterium longum, Bifidobacterium breve, Bifidobacterium lactis, Lactobacillus reuteri, Lactobacillus gasseri, Enterococcus faecium, Clostridium butyricum, Lactobacillus rhamnosus, Streptococcus thermophilus, Lactobacillus delbrueckii ssp. Bulgaricus, Lactobacillus helveticus, Lactobacillus fermentum, Lactobacillus paracasei, Lactobacillus salivarius, Lactococcus lactis, Enterococcus faecalis, Bifidobacterium bifidum, or a combination thereof.

10. The composition of claim 1, wherein the composition is dissolved in a solvent to reactivate the lyophilized probiotics before intake.

11. A lyophilized probiotic product comprising the composition of claim 1.

12. The lyophilized probiotic product of claim 11, wherein the product is to be dissolved in a solvent for reactivating lyophilized probiotics before intake.

13. A method for reactivation of lyophilized probiotics, comprising contacting the lyophilized probiotics with a reactivator, wherein the reactivator comprises at least one selected from the group consisting of L-lysine, L-ornithine, L-tyrosine, and L-histidine.

14. A method for screening a substance function to reactivate lyophilized probiotics, the method comprising selecting a substance conferring a negative zeta-potential on the cell surface of the lyophilized probiotics.

15. The composition of claim 3, wherein the reactivator is contained in such an amount as to form a final concentration of 0.01 M to 0.15 M when dissolved in a solvent.

16. The composition of claim 2, further comprising at least one carbohydrate selected from the group consisting of fructose, sucrose, sorbitol, glucose, maltose, trehalose, and fructooligosaccharide.

17. The composition of claim 16, wherein the carbohydrate is contained in an amount of 0.1 g to 8 g per 10 g of the composition.

18. The composition of claim 2, comprising lyophilized probiotics at a concentration of 1×10⁸ to 1×10¹² CFU/g.

19. The composition of claim 2, wherein the lyophilized probiotics are in a freeze-dried form of Lactobacillus sp., Lactococcus sp., Enterococcus sp., Bifidobacterium sp., Pediococcus sp., Streptococcus sp., or a combination thereof.

20. The composition of claim 19, wherein the lyophilized probiotics are in a freeze-dried form of Lactobacillus plantarum, Lactobacillus acidophilus, Lactobacillus casei, Streptococcus thermophilus, Bifidobacterium animalis, Bifidobacterium longum, Bifidobacterium breve, Bifidobacterium lactis, Lactobacillus reuteri, Lactobacillus gasseri, Enterococcus faecium, Clostridium butyricum, Lactobacillus rhamnosus, Streptococcus thermophilus, Lactobacillus delbrueckii ssp. Bulgaricus, Lactobacillus helveticus, Lactobacillus fermentum, Lactobacillus paracasei, Lactobacillus salivarius, Lactococcus lactis, Enterococcus faecalis, Bifidobacterium bifidum, or a combination thereof.
